(19) United States
(12) Patent Application Publication
(10) Pub. No.: US 2018/0001043 A1
(43) Pub. Date: Jan. 4, 2018

(54) Tracheal protective neonatal ventilation device

(71) Applicant: The Penn State Research Foundation, University Park, PA (US)

(72) Inventor: James Mooney, Hummelstown, PA (US)

(21) Appl. No.: 15/541,046

(22) PCT Filed: Jan. 5, 2016
(86) PCT No.: PCT/US16/12180

§ 371 Date: Jun. 30, 2017

Related U.S. Application Data
(60) Provisional application No. 62/099,794, filed on Jan. 5, 2015.

(51) Int. Cl. A61M 16/04; A61M 16/00

(52) U.S. Cl. CPC A61M 16/0411; A61M 16/0415; A61M 16/0445; A61M 16/0409; A61M 2016/003; A61M 16/0463; A61M 2016/0413; A61M 2205/8206; A61M 2240/00; A61M 2016/0027

(57) ABSTRACT

The present invention relates to a tracheal protective ventilation device specially adapted for use with neonates, a stylet that aids in insertion and spatial verification of the tracheal protective ventilation device, and methods for using the same. The ventilation provided by the devices of the present invention is atraumatic to the trachea and the glottis compared to ventilation devices currently available in the art, and has the additional feature of allowing the administration of nutritional matter to the gut without interrupting airflow.

### CROSS-REFERENCE TO RELATED APPLICATIONS

This application claims priority to U.S. Provisional Patent Application No. 62/099,794 filed Jan. 5, 2015, the contents of which are incorporated by reference herein in their entirety.

### BACKGROUND OF THE INVENTION

Traditional ventilation of neonates with an endotracheal tube relies on a relatively rigid tube going between the vocal cords and the often use of a pressure cuff that bears against the tracheal wall. These tubes can lead to damage to the trachea, glottis, and/or vocal cords in the patient being ventilated with potentially severe long term consequences. Such tubes can also be traumatic and difficult to place.

Neonatal airway management has traditionally relied on endotracheal tubes (ETT), which enter the trachea between the vocal cords, via the nasal or oral passages. While excellent for securing the airway and assisting with ventilation, they are not without potentially dramatic complications. The ETT may be difficult to place correctly, particularly in those with less neonatal/infant airway management experience and when placed correctly they may produce significant changes in the airways, such as tracheal stenosis. In addition to this, they have relatively narrow lumens, minimizing the ability to have the neonate/infant breathe spontaneously and reducing the ability to perform bronchoscopies while maintaining airway control.

There is a need in the art for a ventilation device that enables easy control of neonatal airways in a manner that minimizes risk, dependence on ventilator support, and complications. The present invention addresses this unmet need.

### SUMMARY OF THE INVENTION

The present invention relates to a tracheal protective ventilation device for use with neonates and a method for using the same. In addition, the present invention includes a stylet that aids in insertion and spatial verification of the tracheal protective ventilation device. Furthermore, the ventilation of the present invention is atraumatic to the trachea, the glottis, and the vocal cords compared to ventilation devices currently available in the art, and can also facilitate the administration of nutritional matter to the gut without interrupting airflow.

In one aspect, the invention is a tracheal protective ventilation device comprising a retropharynx cuff with an epiglottal extension, an esophageal cuff, a breathing tube, and at least one inflation tube, wherein the at least one inflation tube is connected to the retropharynx cuff and esophageal cuff. In one embodiment, the epiglottal extension extends from the retropharynx cuff when deflated and rises to lift the neonatal epiglottis, pinning the neonatal epiglottis against the back of the tongue, when inflated.

In one embodiment, the breathing tube includes a proximal end and a distal end, said proximal end comprises a standard breathing circuit adapter, and said distal end comprises an opening for the inflow and outflow of air. In one embodiment, the breathing tube has a length of between 35-75 mm. In one embodiment, the breathing tube has an outer diameter of between 6-8 mm. In one embodiment, the breathing tube has an inner diameter of no more than 1 mm less than the outer diameter.

In one embodiment, the tracheal protective ventilation device further comprises a sensor tube having a proximal end and a distal end, said proximal end comprising a connector for an end-tidal CO₂ monitoring device, said distal end comprising an opening for the sampling of respired air.

In one embodiment, the tracheal protective ventilation device further comprises a feeding tube having a proximal end and a distal end, said proximal end comprising a connector for a feeding tube, said distal end comprising a multi-orifice tube end. In one embodiment, the feeding tube has a length of between 35-60 cm. In one embodiment, the feeding tube has an outer diameter of between 2.5-3 mm. In one embodiment, the feeding tube has an inner diameter of 1.5-2 mm. In one embodiment, the feeding tube is translucent. In one embodiment, the multi-orifice tube end is translucent.

In one embodiment, the at least one inflation tube comprises a proximal end having a connector for a cuff inflator. In one embodiment, the at least one inflation tube inflates and deflates the retropharynx cuff and esophageal cuff simultaneously. In one embodiment, the at least one inflation tube comprises a first inflation tube that inflates and deflates the retropharynx cuff only. In one embodiment, the at least one inflation tube comprises a second inflation tube having a proximal end and a distal end, said proximal end comprising a connector for a cuff inflator, said distal end connecting to the esophageal cuff only and inflating and deflating the esophageal cuff only.

In one embodiment, the esophageal cuff is a single cuff with ridges, fins, or gills. In one embodiment, the esophageal cuff comprises a plurality of cuffs with each cuff forming a ridge, fin, or gill.

In one embodiment, the tracheal protective ventilation device further comprises a flow sensor that communicates with a patient triggered ventilation system. In one embodiment, the tracheal protective ventilation device further comprises at least one pressure monitor. In one embodiment, the tracheal protective ventilation device further comprises at least one electrical sensor.

In another aspect, the invention relates to an illuminating stylet device comprising a finger grip comprising a battery housing, a rod, and a high luminance light source, wherein the battery in the finger grip powers the high luminance light source. In one embodiment, the high luminance light source is positioned at the distal end of the rod. In one embodiment, the high luminance light source is positioned in the finger grip. In one embodiment, the rod comprises an optical fiber that directs light from the high luminance light source to the distal end of the rod. In one embodiment, the rod is translucent. In one embodiment, the rod diffuses light from the high luminance light source along its entire length.

In another aspect, the invention relates to a method for inserting a tracheal protective ventilation device into a neonate, the method comprising: inserting an illuminating stylet into a tracheal protective ventilation device comprising a plurality of tubes, a retropharynx cuff comprising an epiglottal extension, and an esophageal cuff; inserting the tracheal protective ventilation device into at least a portion of the upper airway of a neonate; guiding the illuminating stylet and one of the plurality of tubes of the tracheal protective ventilation device into the esophagus of a neonate such that the illumination is visible from the abdomen of the neonate; inflating the retropharynx cuff such that the epiglottal extension rises to lift the neonatal epiglottis, pinning the neonatal epiglottis against the back of the tongue; inflating the esophageal cuff; and removing the illuminating stylet from the tracheal protective ventilation device.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A and FIG. 1B depict an exemplary ventilation device of the present invention and an exemplary stylet of the present invention.

FIG. 2A through FIG. 2C depict the design of an exemplary retropharynx cuff. FIG. 2A depicts the exemplary retropharynx cuff positioned near the neonatal epiglottis. FIG. 2B depicts the exemplary retropharynx cuff in the process of being inflated, which lifts the epiglottal extension to raise the neonatal epiglottis.

FIG. 2C depicts the exemplary retropharynx cuff fully inflated, with the inflated epiglottal extension pressing the neonatal epiglottis against the back of the tongue.

FIG. 3 depicts an exemplary ventilation device of the present invention being inserted into an infant with the aid of an exemplary stylet of the present invention.

FIG. 4 depicts an exemplary ventilation device deployed within an infant with the stylet removed.

FIG. 5A and FIG. 5B depict alternative ventilation devices. FIG. 5A depicts an alternative ventilation device having a different retropharynx cuff design when fully inflated. FIG. 5B depicts an alternative ventilation device having a retropharynx cuff contacting the epiglottis and the laryngopharyngeal wall.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical ventilation devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### DESCRIPTION

The present invention relates to a ventilation device for use with neonates and a method for using the same. In addition, the present invention includes a stylet that aids in insertion and spatial verification of the ventilation device. The ventilation device of the present invention is atraumatic to the trachea and the glottis compared to ventilation devices currently available in the art, and features an inflatable cuff specially designed to accommodate the longer and floppier epiglottis of the neonate. The ventilation device of the present invention has the additional feature of allowing the administration of nutritional matter to the gut without interrupting airflow.

### Tracheal Protective Ventilation Device

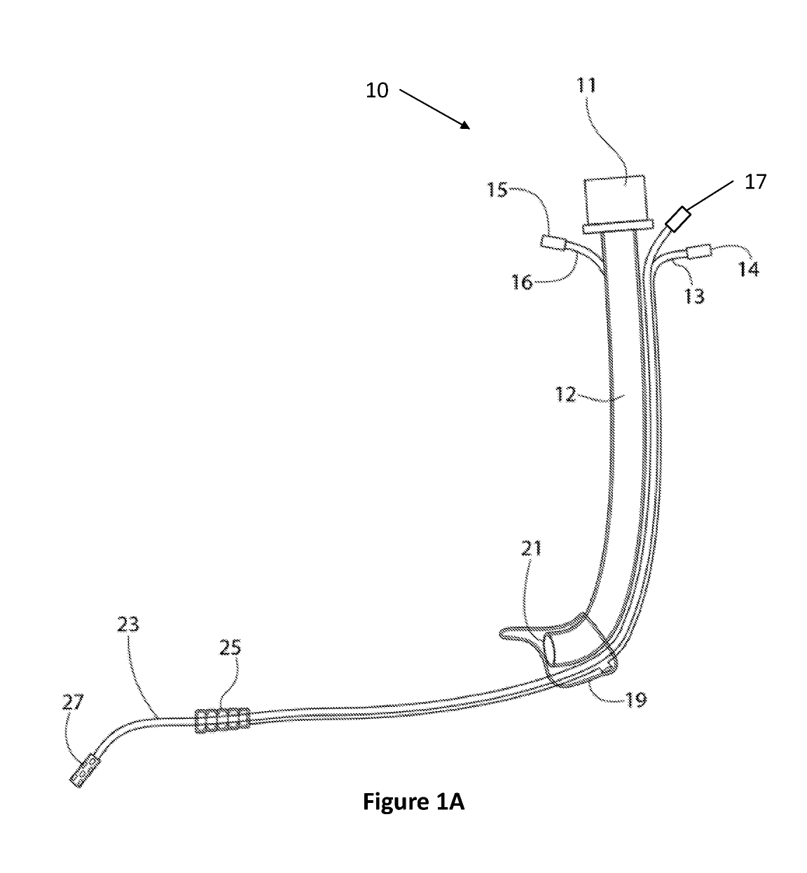

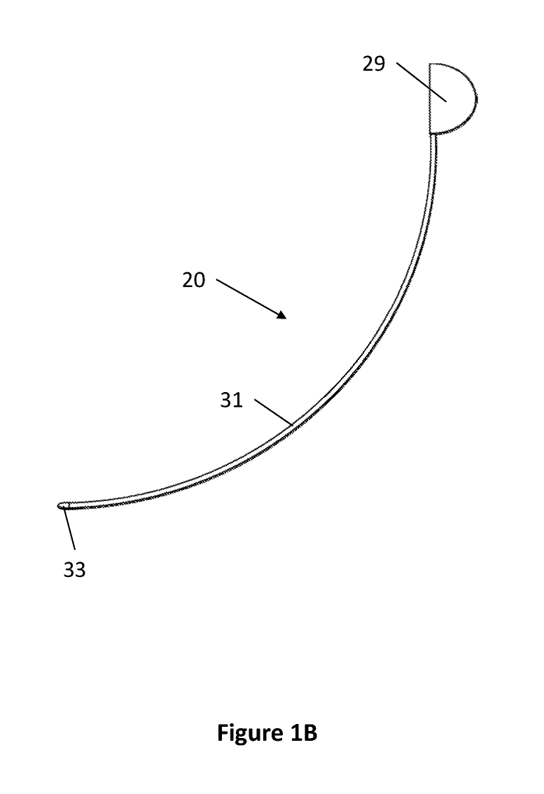

Referring now to FIG. 1A, an exemplary embodiment of a ventilation device 10 of the present invention is shown. Ventilation device 10 comprises a first tube or breathing tube 12, having a lumen for directing the inflow and outflow of air to and from a patient; a second tube or sensor tube 16, having a connector 15 for an end-tidal CO₂ monitoring device; a third tube or feeding tube 23, having a lumen for gastric access and stylet guidance; and inflation tube 13, having a lumen for inflating and deflating retropharynx cuff 19 and esophageal cuff 25.

First tube 12 comprises an opening at its proximal end 11, where a standard breathing circuit adapter allows for the attachment of any suitable breathing circuit commonly used in the art. First tube 12 also comprises an open ended distal end 21 that allows for the inflow and outflow of air. Second tube 16 is positioned such that it extends along the length of first tube 12 and terminates with an opening by the distal end 21 of first tube 12 to allow for an end-tidal CO₂ monitoring device attached to connector 15 to sample respired air for proper placement determination, gross cardiac output, and respiratory changes (capnography). Third tube 23 is positioned such that it extends along the length of first tube 12 but is dimensioned to be longer than first tube 12. Third tube 23 comprises a connector 17 for nutrient and stylet access and terminates in multi-orifice end 27 to mitigate occlusion from administered nutrients and stomach contents.

Inflation tube 13 comprises a standard proximal connector 14 for the attachment of any suitable cuff inflator commonly used in the art, such as a syringe or a manometer inflator device. In some embodiments, inflation tube 13 is positioned such that it extends along the length of first tube 12 to branch into a first opening into retropharynx cuff 19, and continues along the length of third tube 23 to branch into a second opening into esophageal cuff 25. In this configuration, the inflow and outflow of air via inflation tube 13 inflates and deflates retropharynx cuff 19 and esophageal cuff 25 simultaneously. In some embodiments, inflation tube 13 is positioned such that it extends along the length of first tube 12 to terminate into a first opening into retropharynx cuff 19. In some embodiments, a second inflation tube is positioned such that it begins at the proximal end of the device and extends along the length of third tube 23 to terminate into a second opening into esophageal cuff 25 (not pictured). In this configuration, the inflow and outflow of air via inflation tube 13 inflates and deflates retropharynx cuff 19 alone. In the same configuration, the inflow and outflow of air via the second inflation tube inflates and deflates esophageal cuff 25 alone.

First tube 12, second tube 15, third tube 23, and inflation tube 13 may be composed of a flexible thermoplastic material, such as polyvinyl chloride, or a flexible non-thermoplastic material, such as silicon. The materials may be translucent or opaque. The proximal connectors of each tube may be composed of any conventional material known in the art, such as polyvinyl chloride or polyurethane. The proximal connectors of each tube may comprise different lengths to allow for easy access and prompt identification. The proximal connectors of each tube may comprise different coloring for prompt identification. First tube 12, second tube 15, third tube 23, and inflation tube 13 may comprise depth markers to aid the operator of the device in determining the position of the device in the case of blind insertion. Multi-orifice end 27 is preferably translucent. In some embodiments, first tube 12 has a length of between 35-75 mm, an outer diameter of between 6-8 mm, and an inner diameter of no more than 1 mm less than the outer diameter. In some embodiments, third tube 23 has a length of between 35-60 cm, an outer diameter of between 2.5-3 mm, and an inner diameter of 1.5-2 mm.

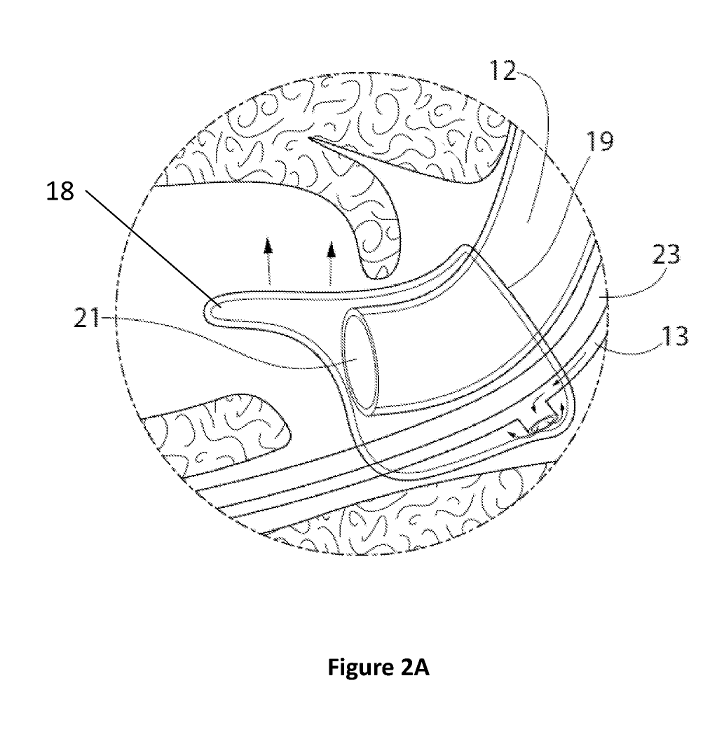

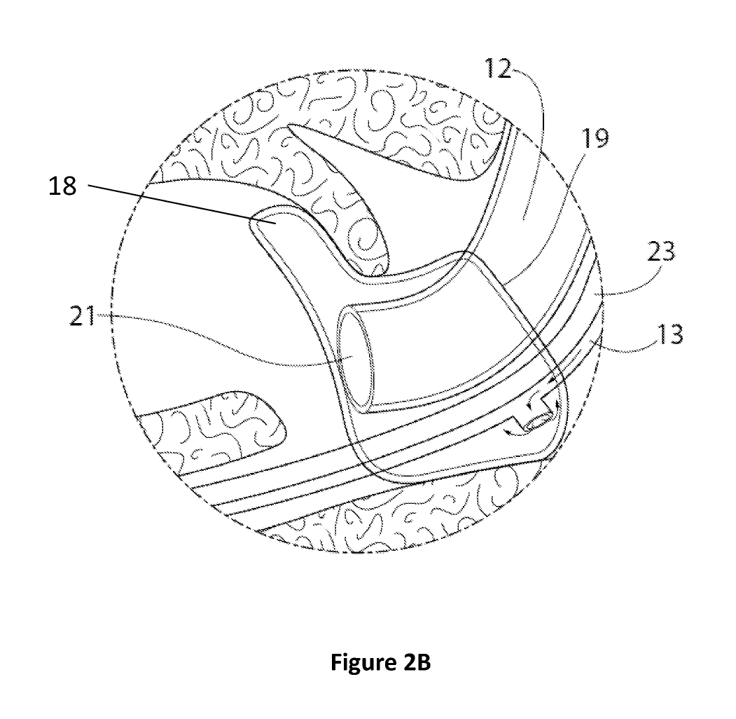

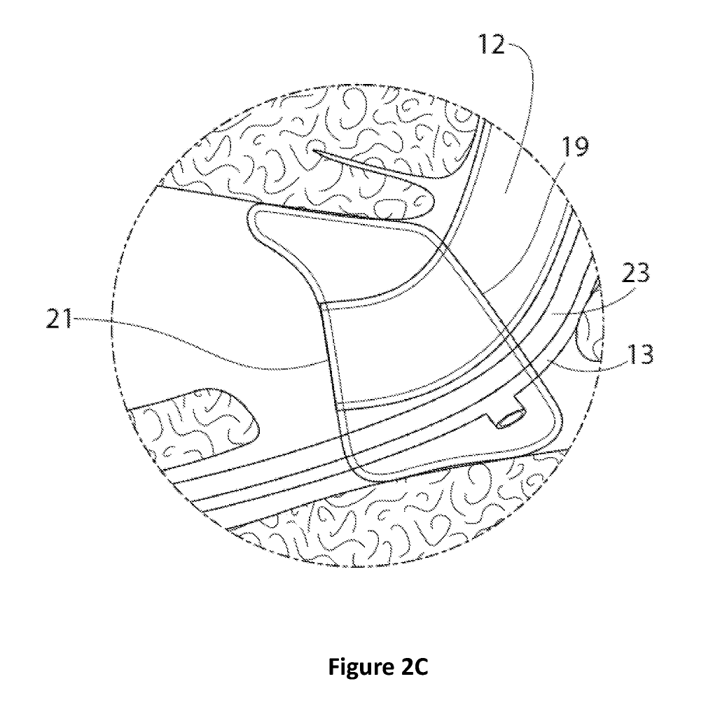

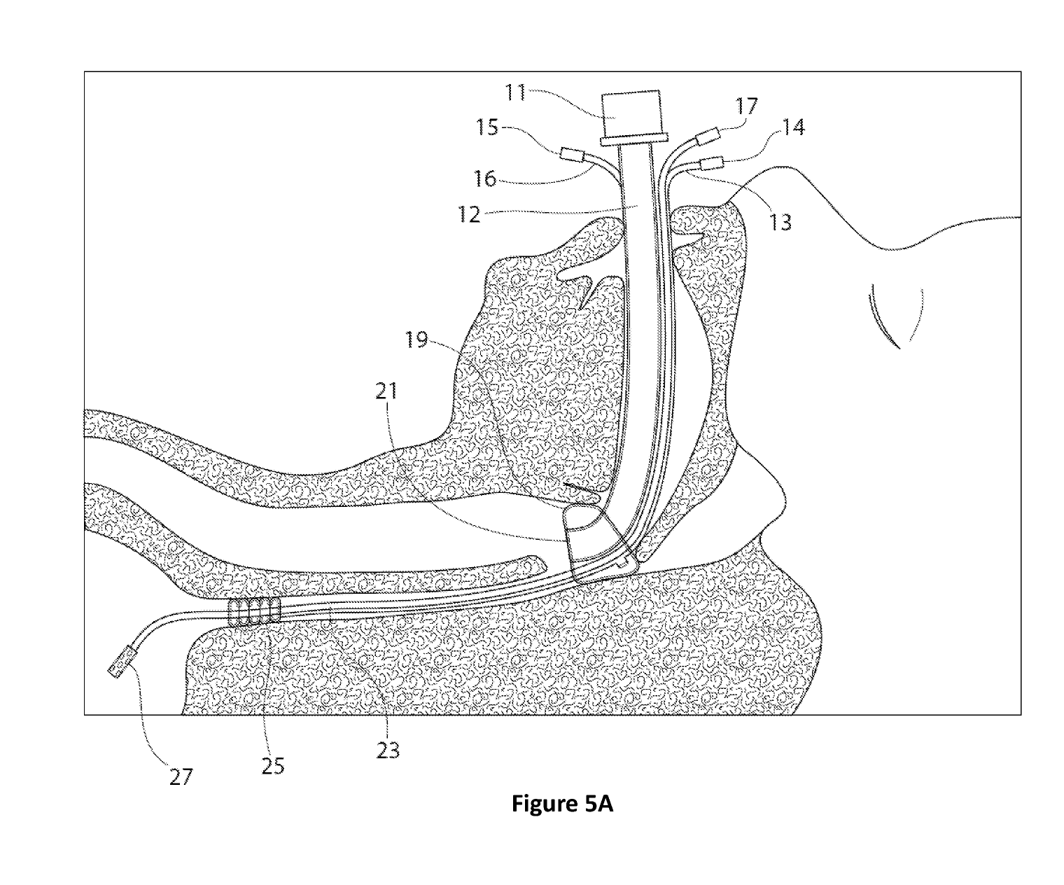

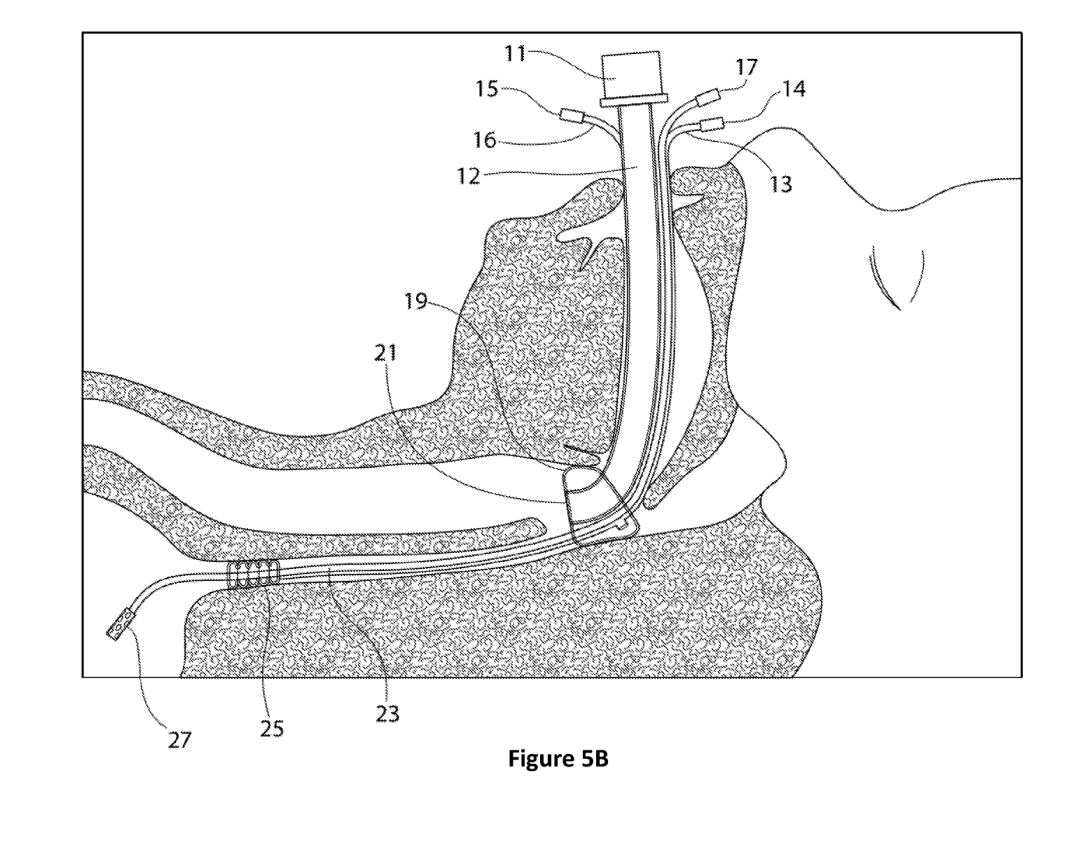

Retropharynx cuff 19 is specially designed for the unique anatomy of the neonate. The epiglottis of the neonate is longer and floppier than in older patients. As a result, when the neonate is in a supine position, the epiglottis drops down and partially occludes the tracheal opening. Referring now to FIG. 2A through FIG. 2C, an exemplary retropharynx cuff 19 is depicted deflated (FIG. 2A), partially inflated (FIG. 2B), and fully inflated (FIG. 2C). Retropharynx cuff 19 comprises epiglottal extension 18 to accommodate the longer and floppier epiglottis of the neonate. Epiglottal extension 18 also increases the area of contact between retropharynx cuff 19 and the floppy epiglottis, providing a significant improvement over the art in that it ensures consistent and proper lift of the floppy epiglottis and prevents the floppy epiglottis from uncomfortable folding or curling. When retropharynx cuff 19 is deflated, epiglottal extension 18 is in a collapsed form (FIG. 2A). In some embodiments, epiglottal extension 18 is held close to first tube 12, such that epiglottal extension 18 does not snag during insertion of the ventilation device. Epiglottal extension 18 can be held close to first tube 12 using any suitable mechanism, such as a weak elastic band or a flexible strip of material. As retropharynx cuff 19 is inflated, epiglottal extension 18 rises to catch and lift the floppy epiglottis of the neonate, clearing the tracheal opening (FIG. 2B). When retropharynx cuff 19 is fully inflated, epiglottal extension 18 presses against the floppy epiglottis of the neonate to pin it against the back of the tongue, maintaining clearance to the tracheal opening (FIG. 2C). Persons skilled in the art will appreciate that the fully inflated conformation of retropharynx cuff 19 may be any shape suitable for providing an airtight fit within the throat region, such as a shape having a circular cross section to accommodate the width of the throat, or an alternative cuff shape such as the one depicted in FIG. 5A.

Retropharynx cuff 19 and esophageal cuff 25 may be composed of latex or an elastic material such as polyvinyl chloride. In some embodiments, retropharynx cuff 19 further comprises regions of varying material thickness or rigidity. For example, epiglottal extension 18 may comprise a thicker or more rigid material, such that during inflation, epiglottal extension 18 remains substantially planar to uniformly lift the neonatal epiglottis. Similarly, other regions that are desired to expand more readily may be composed of thinner material to promote greater elasticity in those regions of the cuff Retropharynx cuff 19 and esophageal cuff 25 may be adhered to the device by any appropriate means known in the art, such as by a cyanoacrylate adhesive or a thermal weld.

In some embodiments, retropharynx cuff 19 has high volume and low pressure. Retropharynx cuff 19 can be inflated below 25 mmHG, and can tolerate pressures of at least 30 mmHG. In some embodiments, retropharynx cuff 19 seals the opening of the airway from the oropharynx when inflated. In some embodiments, esophageal cuff 25 has high volume and low pressure. Esophageal cuff 25 can be inflated below 25 mmHG, and can tolerate pressures of at least 30 mmHG. In some embodiments, esophageal cuff 25 is a single cuff with ridges, fins, or gills to take advantage of esophageal motility to secure the cuff and effectively seal the esophagus. In some embodiments, esophageal cuff 25 comprises a plurality of cuffs, wherein each cuff forms an individual ridge, fin, or gill to take advantage of esophageal motility to secure the cuff and effectively seal the esophagus.

### Illuminating Stylet

Another aspect of the invention is stylet 20. Referring now to FIG. 1B, an exemplary stylet 20 is depicted. Stylet 20 comprises finger grip 29, rod 31, and distal tip 33. In some embodiments, the construction of rod 31 may comprise semi-flexible materials including, but not limited to: polyvinyl chloride, silicone, polyethylene, and the like. Rod 31 is dimensioned such that it is at least as long as the length of third tube 23 with a diameter smaller than the lumen of third tube 23 such that rod 31 may fit within the lumen of third tube 23.

In some embodiments, finger grip 29 houses a battery and a high luminance light source, such as an LED light or any other light source known in the art. In a first embodiment where a high luminance light source is housed in finger grip 29, rod 31 may comprise an optical fiber that directs the light to distal tip 33, where it is diffused. In a second embodiment where a high luminance light source is housed in finger grip 29, rod 31 may alternatively be translucent such that rod 31 diffuses light along its entire length. In other embodiments, the high luminance light source is housed in distal tip 33. In a third embodiment where a high luminance light source is housed in distal tip 33, rod 31 may house or be in part composed of conductive material necessary to power the high luminance light source in distal tip 33 using the battery housed in finger grip 29.

In the first and third embodiments of stylet 20, the luminance of distal tip 33 is visible through a translucent multi-orifice end 27 and through the anatomy of the patient to allow the operator of the device to visually judge the position of distal tip 33 within the patient during insertion, enabling high confidence in correct placement. In the second embodiment of stylet 20, the luminance of rod 31 is visible through a translucent third tube 23 and through the anatomy of the patient to allow the operator of the device to visually judge the position of rod 31 within the patient during insertion, enabling high confidence in correct placement. An operator using stylet 20 to insert a ventilation device may determine that placement of the device is incorrect due to illumination emanating from the patient's trachea. Furthermore, an operator may determine that placement of the device is correct due to illumination emanating from the patient's esophagus or abdomen.

### Methods of Use

The anatomy of a neonate's airway features a lower hanging uvula and soft palate and an epiglottis that angled away from the axis of the trachea that is longer, more anterior and higher than an adult's. The neonatal glottis is higher and closer to the head, at about the C1 and C2 vertebrae of the spine, whereas the adult glottis is located nearer to the C3 and C4 vertebrae. As a result, extreme angles are involved when attempting to access the trachea of a neonate. The present invention provides methods of using the systems and devices of the present invention to avoid the need of accessing the neonatal trachea by sealing the upper pharynx of the patient.

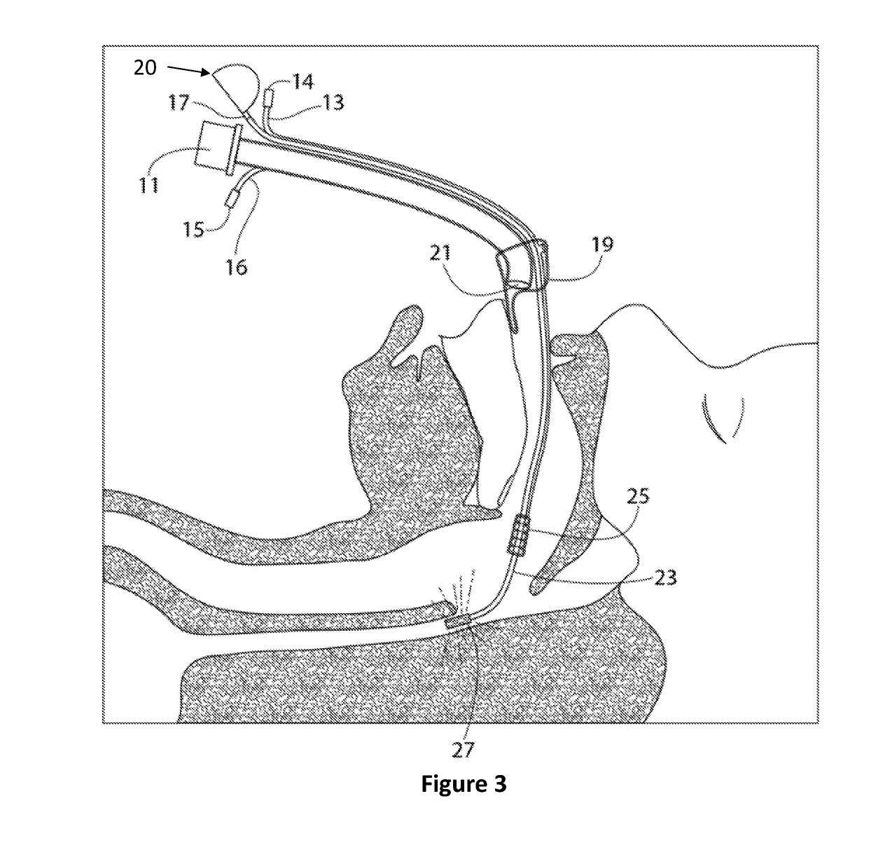

Referring now to FIG. 3, an exemplary ventilation device of the present invention is depicted being inserted into a neonate with the aid of an exemplary stylet of the present invention. Prior to insertion, stylet 20 is first placed in the lumen of third tube 23 to provide the ventilation device with rigidity and with illumination emanating from multi-orifice end 27. The operator inserts a finger or a thumb into the mouth of the neonate to open the mouth and lift the mandible and soft tissues. The ventilation device and stylet are passed into at least a portion of the upper airway of the neonate, where it reaches the posterior wall of the oropharynx. The operator then passes the device and stylet into the esophagus and finally the stomach. Illumination from the distal tip of stylet 20 is visible from within multi-orifice end 27 to help the operator determine correct placement. For example, illumination through the abdomen wall indicates correct placement, while illumination from the anterior aspects of the trachea indicates incorrect placement. At any time after correct placement has been determined, stylet 20 may be removed.

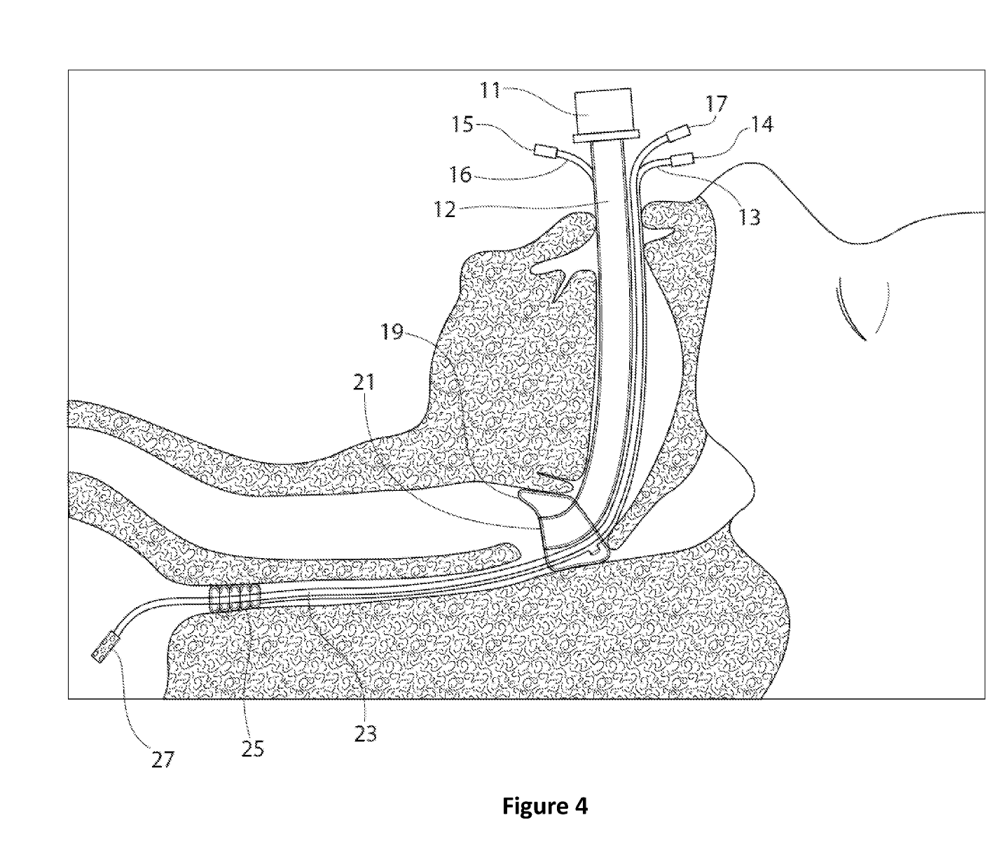

When the ventilation device is properly inserted, distal end 21 of first tube 12 is situated in the upper pharynx and multi-orifice end 27 of third tube 23 is situated in the stomach (FIG. 4). Retropharynx cuff 19 is inflated to lift up the floppy epiglottis with epiglottal extension 18, pinning the floppy epiglottis against the back of the tongue. In some embodiments, a fully inflated retropharynx cuff 19 secures the device by contacting the epiglottis, laryngopharyngeal wall, and soft palate of the upper airway (FIG. 4). In other embodiments, a fully inflated retropharynx cuff 19 secures the device by contacting only the epiglottis and the laryngopharyngeal wall (FIG. 5B). Esophageal cuff 25 is inflated to secure the device by contacting the esophageal wall, preferably in the area of the second segment of the esophagus, where peristaltic activity develops earliest. In this configuration, an airtight seal is formed to prevent air from escaping through the esophagus or retropharynx and allows air that is transported into the laryngopharynx via first tube 12 to enter and exit the trachea and lungs. Since no part of the device is physically in the trachea of the patient, there is less chance for tissue necrosis (due to pressure on tissue), trauma and scarring (due to rigid edges of devices being introduced) and abnormal development (due to non-anatomic pressure on developing tissues). There is also a reduced chance of respiratory infections (less ventilator dependent time), cardiac instability and barotrauma (due to the negative consequences of positive pressure ventilation).

The device does not enter the trachea so the inner and outer diameters of first tube 12 can be large to provide lower airway resistance, to diminish the likelihood of airway occlusion, to decrease the work of breathing, and to cause less fatigue in the muscles of respiration. The large diameter of first tube 12 also make first tube 12 amenable to access by other instruments via standard adapters, such as a bronchoscope for imaging of the lungs and free functioning glottis, aspiration of secretions, or placement of surfactants. The device may be kept in a patient for extended periods of time by allowing gastric administration of nutrients and fluids via third tube 23. In some embodiments, the device may include a flow sensor for use with a patient triggered ventilation system. In some embodiments, the device may include pressure monitoring devices for monitoring, for example, esophageal pressure or gastric pressure in patients undergoing diaphragmatic pacing. In some embodiments, the device may include electrical wiring for sensing native diaphragm activity (to trigger ventilation devices) or to pace local tissue such as the diaphragm (to trigger breaths) or esophageal/intestinal activity.

### Kits

The invention also includes a kit comprising components useful within the methods of the invention and instructional material that describes, for instance, the method of using the tracheal protective ventilation devices and illuminating stylet devices as described elsewhere herein. In certain embodiments, the kit comprises instructional material that details the methods of the invention, such as the method of inserting the illuminating stylet device into the tracheal protective ventilation device to aid in insertion into the patient. Instructional material may include a publication, a recording, a diagram, or any other medium of expression which can be used to communicate the usefulness of the device described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A tracheal protective ventilation device comprising:
a retropharynx cuff with an epiglottal extension;
an esophageal cuff;
a breathing tube; and
at least one inflation tube, wherein the at least one inflation tube is connected to the retropharynx cuff and esophageal cuff.

2. The tracheal protective ventilation device of claim 1, wherein the epiglottal extension extends from the retropharynx cuff when deflated and rises to lift the neonatal epiglottis, pinning the neonatal epiglottis against the back of the tongue, when inflated.

3. The tracheal protective ventilation device of claim 1, wherein the breathing tube includes a proximal end and a distal end, said proximal end comprises a standard breathing circuit adapter, and said distal end comprises an opening for the inflow and outflow of air.

4. The tracheal protective ventilation device of claim 3, wherein the breathing tube has a length of between 35-75 mm.

5. The tracheal protective ventilation device of claim 3, wherein the breathing tube has an outer diameter of between 6-8 mm.

6. The tracheal protective ventilation device of claim 3, wherein the breathing tube has an inner diameter of no more than 1 mm less than the outer diameter.

7. The tracheal protective ventilation device of claim 1, further comprising a sensor tube having a proximal end and a distal end, said proximal end comprising a connector for an end-tidal CO₂ monitoring device, said distal end comprising an opening for the sampling of respired air.

8. The tracheal protective ventilation device of claim 1, further comprising a feeding tube having a proximal end and a distal end, said proximal end comprising a connector for the feeding tube, said distal end comprising a multi-orifice tube end.

9. The tracheal protective ventilation device of claim 8, wherein the feeding tube has a length of between 35-60 cm.

10. The tracheal protective ventilation device of claim 8, wherein the feeding tube has an inner diameter of between 1.5-2 mm.

11. The tracheal protective ventilation device of claim 8, wherein the feeding tube has an outer diameter of between 2.5-3 mm.

12. The tracheal protective ventilation device of claim 8, wherein the feeding tube is translucent.

13. The tracheal protective ventilation device of claim 8, wherein the multi-orifice feeding tube end is translucent

14. The tracheal protective ventilation device of claim 1, wherein the at least one inflation tube comprises a proximal end having a connector for a cuff inflator.

15. The tracheal protective ventilation device of claim 14, wherein the at least one inflation tube inflates and deflates the retropharynx cuff and esophageal cuff simultaneously.

16. The tracheal protective ventilation device of claim 14, wherein the at least one inflation tube comprises a first inflation tube that inflates and deflates the retropharynx cuff only.

17. The tracheal protective ventilation device of claim 1, wherein the at least one inflation tube comprises a second inflation tube having a proximal end and a distal end, said proximal end comprising a connector for a cuff inflator, said distal end connecting to the esophageal cuff only and inflating and deflating the esophageal cuff only.

18. The tracheal protective ventilation device of claim 1, wherein the esophageal cuff is a single cuff with ridges, fins, or gills.

19. The tracheal protective ventilation device of claim 1, wherein the esophageal cuff comprises a plurality of cuffs with each cuff forms a ridge, fin or gill.

20. The tracheal protective ventilation device of claim 1, further comprising a flow sensor that communicates with a patient triggered ventilation system.

21. The tracheal protective ventilation device of claim 1, further comprising at least one pressure monitor.

22. The tracheal protective ventilation device of claim 1, further comprising at least one electrical sensor.

23. An illuminating stylet device comprising:
a finger grip comprising a battery housing;
a rod; and
a high luminance light source;
wherein the battery in the finger grip powers the high luminance light source.

24. The illuminating stylet device of claim 23, wherein the high luminance light source is positioned at the distal end of the rod.

25. The illuminating stylet device of claim 23, wherein the high luminance light source is positioned in the finger grip.

26. The illuminating stylet device of claim 25, wherein the rod comprises an optical fiber that directs light from the high luminance light source to the distal end of the rod.

27. The illuminating stylet device of claim 25, wherein the rod is translucent.

28. The illuminating stylet device of claim 27, wherein the rod diffuses light from the high luminance light source along its entire length.

29. A method for positioning a tracheal protective ventilation device into a neonate, the method comprising:
inserting an illuminating stylet into a tracheal protective ventilation device comprising a plurality of tubes, a retropharynx cuff comprising an epiglottal extension, and an esophageal cuff;
inserting the tracheal protective ventilation device into at least a portion of the upper airway of a neonate;
guiding the illuminating stylet and one of the plurality of tubes of the tracheal protective ventilation device into the esophagus of a neonate such that the illumination is visible from the abdomen of the neonate;
inflating the retropharynx cuff such that the epiglottal extension rises to lift the neonatal epiglottis, pinning the neonatal epiglottis against the back of the tongue;
inflating the esophageal cuff; and
removing the illuminating stylet from the tracheal protective ventilation device.
